(19) United States
(12) Patent Application Publication
(10) Pub. No.: US 2022/0304693 A1
(43) Pub. Date: Sep. 29, 2022

(54) DEVICE AND METHOD FOR ATTACHING A THREAD LOOP AROUND A SKIN TAG

(71) Applicant: Scandinavian Health Trade AB, Sundbyberg (SE)

(72) Inventors: Gustaf EKLUND, Mariehamn (FI); David WIDLUND, Sundbyberg (SE)

(21) Appl. No.: 17/640,415

(22) PCT Filed: Aug. 21, 2020
(86) PCT No.: PCT/SE2020/050804

§ 371 Date: Mar. 4, 2022

(30) Foreign Application Priority Data
Sep. 11, 2019 (SE) SE 1930292-6

(51) Int. Cl. A61B 17/12

(52) U.S. Cl. CPC A61B 17/12009; A61B 2017/00761

(57) ABSTRACT

A device for applying a thread loop around a skin tag (26), the device comprising a thread cartridge (10) arranged to be connected to a tool (1) which has a hollow cylindrical handle (2) with a piston (7) which can be run inside the handle, wherein the piston (7 first end (9) which in a base position of the piston connects to an opening (5) in a front end (3) of the handle, where a thumb grip (16) is attached to the piston (7) and via a slot (17) in the handle (2) is arranged accessible from the outside of the handle whereby the piston (7) is displaceable from its base position along the interior of the handle (2), where the piston (7) has a carrier (18) at its first end (9) to which an inner end of said thread cartridge (10) is arranged to be connected, further a thread loop (14) in an outer end of the thread cartridge (10) is arranged to be laid around the skin tag (26), where a displacement of the piston (7) from the base position by means of the thumb grip (16) contracts the thread loop (14) around the skin tag (26) and the inner end of the thread cartridge (10) is arranged to be detached from the carrier (18) when the handle (2) is tilted in relation to the longitudinal direction of the thread cartridge (10), whereby the handle (2) is removed from the thread cartridge (10).

### TECHNICAL FIELD

The present invention relates to a device being used to attach a contracted thread loop around an undesirable skin tag, whereby the skin tag is strangled, dies and falls off after a time.

### PRIOR ART

Skin tags are here used as the name of the skin growths which we can all get a bit here and there on the body, usually in the face, the neck, arm pits or groins. These skin tags are also called fibroma (acrochorda) and consist of connective tissue cells and collagen. They can be removed surgically by being cut off, burned away usually by means of alternating current, or by means of the application of active specific liquids. Skin tags are harmless but can be perceived as unpleasant. In this specification the term skin tag is also meant to include teardrop-shaped birthmarks as well as old age warts.

Prior art also includes mechanical devices where a user can choke the skin tag by means of a wire snare which is positioned around the base of the skin tag or by means of a loop being applied around the skin tag so that it, due to lack of nutrition, dies and falls off the skin. Such known technology is disclosed in, for example, the patent specification US 2017273705. In this text, a tool is reported in which a snare is tightened around a skin tag. A user pulls a ring connected to the snare. Due to this construction, the tool is not so convenient for being handled with only one hand, whereby it is not so well suited for a user who uses the tool on his/her own body. Furthermore, fitting the wire for use of the tool is not straightforward.

Another example of the prior art is disclosed in the publication US 2018092646. Here, an aid is described which comprises a thread snare which is wrapped around a skin tag. The thread has loose ends by means of which a user tightens the snare. This kind of aid is also not the easiest to handle for a user, especially when a user uses the aid for removing a skin tag on his own body.

As a further example of prior art, reference is made to the publication GB 2322802. In this is described a tool in which a loop of an elastic material is used being applied around the skin tag. In this case the loop is applied around the skin tag and passed over it by pushing the tool with its front part against and around the skin tag (i.e. against the skin on which the skin tag is located), after which the elastic loop is pressed over and around the skin tag.

### DESCRIPTION OF THE INVENTION

The device according to one aspect of the invention consists of a tool for applying a thread loop around a skin tag, the tool comprising a hollow cylindrical handle with a piston being arranged to run inside the handle, the piston having a first end which in a base position for the piston abuts against an opening in a front end of the handle. A thumb grip is attached to the piston and via a slot in the handle the piston is arranged accessible from the outside of the handle whereby the piston is displaceable from its base position along the interior of the handle. The piston has at a first end a carrier to which an inner end of a thread cartridge is arranged to be connected. A thread loop is arranged to be laid around the skin tag at an outer end of the thread cartridge. A displacement of the piston from the base position by means of the thumb grip concentrates the thread loop around the tag, when the piston with its carrier is displaced backwards from the base position of the piston and thereby stretches the thread when the thread of the thread cartridge is attached to the carrier. The inner end of the thread cartridge is arranged to be detached from the carrier when the handle is tilted in relation to the longitudinal direction of the thread cartridge, thereby slackening the thread, after which the handle is removed from the thread cartridge. The loop of the thread cartridge is then tightened around the skin tag and stops the supply of nutrients to it. The thread cartridge is left at the skin tag and falls off when the skin tag eventually loosens from the skin. It should be mentioned here that the thread cartridge is intended to form part of the device.

It should be mentioned here that the device is arranged to apply a tightened thread loop around a skin tag which has a narrow base so that the thread loop can be effectively applied around the skin tag without sliding away from it. For this reason, the device should be used only for the removal of skin tags (acrochorda) including also teardrop-shaped birthmarks and old age warts (seborrheic keratosis).

A spring inside the handle abuts a rear wall of the handle and presses against the other end of the piston so that the piston is pressed with a predetermined force against the opening in the front end of the handle. In this way a counterforce can be sensed by the user when the user withdraws the piston by moving the thumb grip backwards to tension the thread loop.

The thread cartridge is a pre-formed part which comprises a thread which at its front end has a tightenable loop when the rear part of the thread is pulled through a loop knot in front of which the loop is formed. At its rear end, the thread is provided with a knob being attached to the end of the thread. This knob is placed inside the wall of the carrier, whereby the thread is pulled into the handle when the piston is moved backwards from its base position. Around the thread, between the loop in its front part and the knob in its rear part, a hollow cylinder is arranged, where a through-hole is arranged along its axis. The thread of the thread cartridge can be displaced longitudinally along the through hole.

The carrier in the piston is arranged with a slot in its front wall. The purpose of this slot when loading the tool with the thread cartridge is to be able to insert the thread knob behind the wall of the carrier and to ensure that the thread can be advanced inside the slot and stretched mainly along the longitudinal axis of the handle in front of the handle.

The handle is at the far end of its front end equipped with a clamp with wings that has a longitudinal extension along the handle. When the thread cartridge is loaded to the handle and its piston, the cylinder is pressed down between said wings and becoming hold in position by the wings. Furthermore, a lug is formed next to the clamp behind it, whereby the cylinder cannot slide backwards when tensioning the thread in the thread cartridge when the piston is moved backwards, since the cylinder is stopped by the lug. As a result, each movement backwards of the thumb grip will bring the piston with its carrier backwards and then tighten the loop by pulling thread inwards into the handle.

The device has characteristics according to the features specified in claim 1. Characteristics of a method are specified in the independent method claim. Further embodiments of the invention are presented in the dependent claims.

### BRIEF DESCRIPTION OF THE DRAWINGS

FIG. 1 schematically shows a plan view of the device according to one aspect of the invention.

FIG. 2 depicts in a perspective view a thread cartridge intended for connection to the tool when using it for its purpose.

FIG. 3 shows a longitudinal section of the tool according to FIG. 1.

FIG. 4 shows details of the device and their mutual positions in relation to each other.

FIG. 5 illustrates the front part of the tool with its thread cartridge connected.

FIG. 6 shows a plan view of a thread cartridge and a longitudinal section through the thread cartridge.

FIG. 7 illustrates how the thread cartridge is released from the handle by moving the thumb grip forward and thereby slackening the thread so that it can slide out of the gap in the front of the carrier.

### DESCRIPTION OF EMBODIMENTS

In the following, a number of embodiments of the invention are described with reference to the accompanying drawings. The drawings show only schematically the principle of the device and do not claim to show to any scale any proportions between different elements thereof.

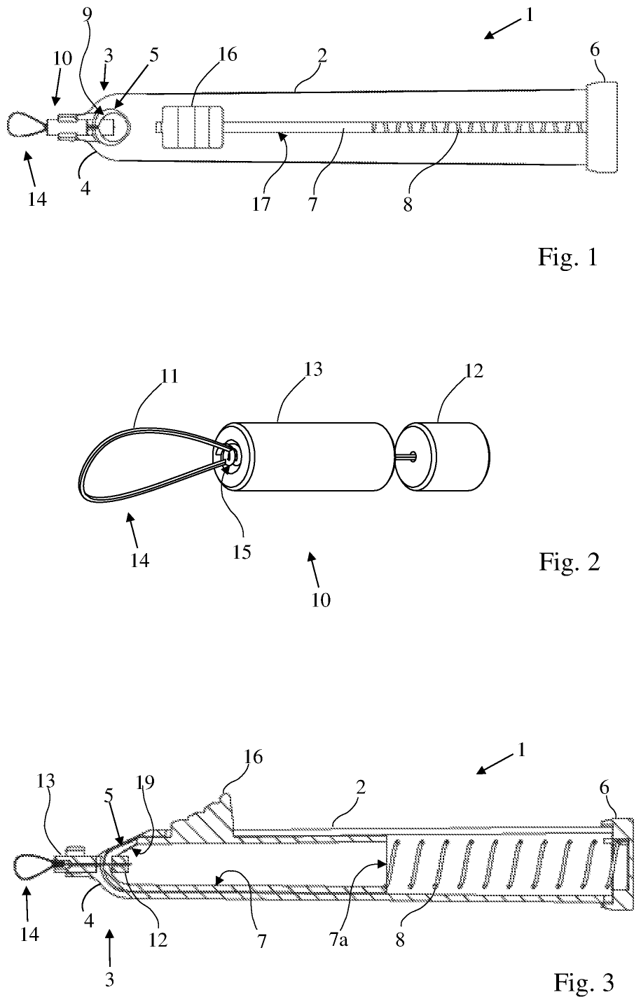

By reference to FIG. 1, the tool 1 according to the aspect of the invention is shown. The tool has a handle 2 which consists of a hollow cylindrical body. Cylindrical means any type of cylinder that has a cross section with a closed curve. In the simplest embodiment, the handle 2 is circular-cylindrical. At its front end 3, the handle has a front wall 4 with an opening 5 which is an oblique cut-out across the handle housing next to the central longitudinal axis of the handle. At the rear end of the handle 2 this has a rear wall 6. The handle 2 has inside a cylindrical longitudinal cavity with preferably the same shape as the outside of the handle. A piston 7 is arranged in this cavity. This piston is arranged to have an external shape corresponding to the shape of the cavity, wherein the piston 7 can run longitudinally inside the handle 2. The piston 7 can be solid or designed hollow in a corresponding manner as the handle. The piston 7 is shorter than the cylindrical cavity of the handle. Between the piston 7 and the rear wall 6 of the handle, a spring 8, for example a helical spring, is arranged. The piston 7 is thus displaceable from a base position, where the first end 9 of the piston abuts against the front wall 4 of the handle 2, to a position rearwards where the spring 8 is completely compressed. The spring 8 abuts against a second end 7a, i.e. towards the rear end of the piston 7.

The thread cartridge 10 of the device is shown in detail in FIG. 2. It appears that the thread cartridge has a thread 11 which is attached to a knob 12 at the rear. The thread 11 can run freely in a thread channel longitudinally through the hollow cylinder 13. At the front the thread 11 is formed into a tightenable loop 14. The loop knot 15 for the loop is glimpsed at the front of the cylinder 13. The loop knot 15 cannot be pulled into the cylinder 13, since a constriction (see FIG. 6) inside the thread channel in the cylinder prevents this. Thus, if the knob 12 is moved backwards while the cylinder 13 is held still, the loop 14 will be tightened against the loop knot 15.

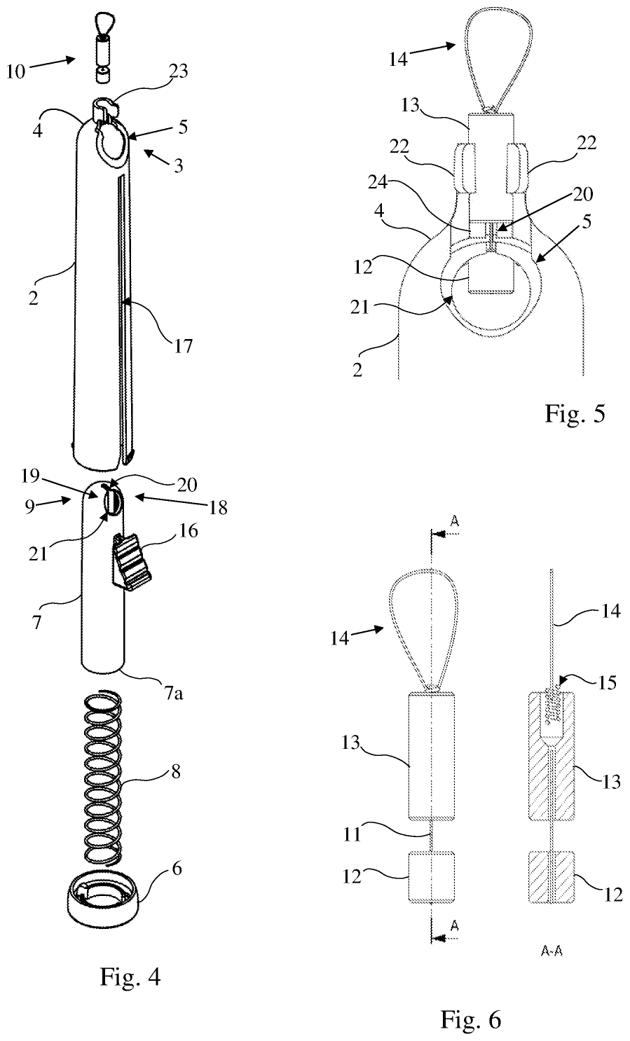

The piston 7 is clearly shown in FIG. 4. Here it is shown with a thumb grip 16. This thumb grip has closest to the body of the piston 7 an elongate bracket which can run along an elongate gap 17 along the housing of the handle 2 in its longitudinal direction, the thumb grip thus being located on the outside of the handle 2 housing. Thus, when the piston 7 is located inside the cavity of the handle 2, the piston 7 can be moved by means of the thumb grip 16 along the gap 17 backwards against the spring 8 and compress this, whereby a counterforce against the movement of the thumb grip is achieved. At the front of the piston 7 at its front end, a carrier 18 is arranged. This carrier 18 consists of a wall 19 which comprises an elongate slot 20 which opens into a hole 21 in the wall 19 of the piston. When loading a thread cartridge 10 in the handle 2, the knob 12 is inserted through the hole 21 so that the knob 12 ends up behind the wall 19 while the thread is lowered into the gap 20, whereby the thread 11 can be pulled backwards and into the handle 2 when the piston 7 is moved backwards with the thumb grip 16.

The loading of a thread cartridge 10 to the handle 2 is most clearly shown in FIG. 5. The knob 12 of the thread cartridge is inserted through the hole 21 so that the knob 12 ends up behind the wall 19 in the piston 7 carrier 18. The thread in front of the knob 12 is lowered into the slot 20. The cylinder 13 is pressed down between the wings 22 at the foremost part of the handle 2. The wings 22 thereby form a clamp 23 which holds the cylinder 13 in position when a thread cartridge is loaded to the handle. Here it is also ensured that the rear wall of the cylinder 13 is arranged in front of an abutment in the form of a lug 24. As a result, the cylinder 13 surrounding the thread 11 cannot be moved backwards when the piston 7 carrier 18 stretches the thread while the piston 7 is displaced backwards by means of the thumb grip 16.

After charging a thread cartridge 10 to the handle 2, a pre-formed thread loop 14 is brought in over a skin tag to be removed from the skin of a client. This can of course also be done by the client himself. The thread loop 14 is laid around the skin tag at its base. After this the thumb grip 16 is displaced backwards along the handle. As a result, as shown above, the thread loop will be tightened around the skin tag. An advantage of this tool is that it can be handled with just one hand by one user.

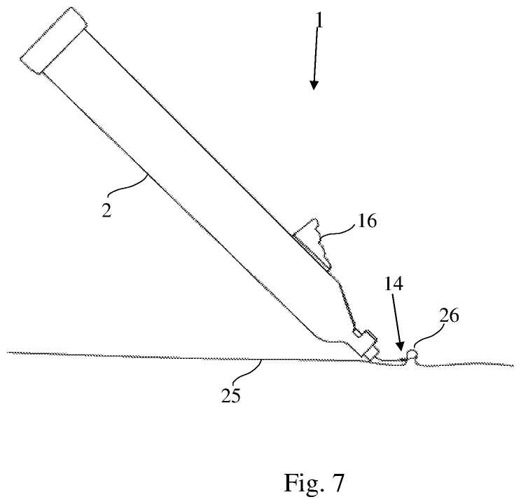

In FIG. 7 it is shown how the thread cartridge 10 is detached from the handle 2. By the user releasing the grip on the handle 2, the thread 11 in the thread cartridge is slackened, since the spring 8 then pushes the piston 7 inside the handle 2 forwards. The user tilts at a large angle, as a suggestion 30 to 90 degrees, up the handle from the skin 25 and suitably presses the front part of the handle against the skin 25 on which the skin tag 26 is located and pulls the front part of the handle in the direction away from the skin tag. As a result, the cylinder 13 of the thread cartridge is detached from the clamp 23 and the thread 11 together with the knob 12 slides out of the carrier 18 due to the inclination of the handle in relation to the longitudinal direction of the thread 11 closest to the skin 25.

After detaching the handle from the thread cartridge, the thread cartridge 10 can remain at the skin tag 26 until it falls off. However, if so desired, the thread 11 can be cut off outside the loop knot 15 of the thread loop 14.

The thread cartridge can be marketed on the market in packages of a larger number with, for example, 5 or 10 thread cartridges per package.

1. A device for applying a thread loop around a skin tag, the device comprising a thread cartridge arranged to be connected to a tool having a hollow cylindrical handle with a piston runningly arranged inside the handle, the piston having a first end which in a base position of the piston abuts to an opening in a front end of the handle, wherein:
a thumb grip is attached to the piston and arranged accessible from the outside of the handle via a slot in the handle whereby the piston is displaceable from its base position along the interior of the handle,
the piston has at its first end a carrier to which an inner end of said thread cartridge is arranged to be connected,
a thread loop at an outer end of the thread cartridge is arranged to be laid around the skin tag,
a displacement of the piston from the base position by means of the thumb grip pulls together the thread loop around the skin tag,
the thread cartridge is arranged to be detached from the carrier by releasing the thumb grip whereby the thread of the thread cartridge can be detached from the carrier.

2. The device according to claim 1, wherein the thread cartridge comprises a thread having at its front end said tightenable thread loop in front of a loop knot and at its rear end a knob intended to be fitted inside the wall of the carrier, whereby the thread is pulled into the handle when the piston is moved backwards from its base position, where further around the thread between the thread loop in its front part and the knob in its rear part, a cylinder with a through hole is arranged along its longitudinal axis, whereby the thread in the thread cartridge can be displaced longitudinally along said through hole.

3. The device according to claim 2, wherein the carrier is arranged with a slot in its front wall, wherein the thread is lowered into the slot and the knob of the thread cartridge arranged behind the carrier wall when the thread cartridge is loaded to the tool.

4. The device according to claim 3, wherein the handle outermost of its front end is equipped with a clamp with wings and wherein the cylinder of the thread cartridge is pressed into the clamp between said wings when the thread cartridge is loaded to the handle and its piston and where further a lug is formed next to the clamp behind it, whereby the cylinder cannot slide backwards when tensioning the thread in the thread cartridge when the piston is moved backwards because a rearward movement of the cylinder is then stopped by the lug.

5. The device according to claim 4, wherein a spring is arranged inside the handle, wherein said spring abuts against a rear wall of the handle and thereby presses against the second end of the piston so that the piston is pressed with a predetermined force against the opening at the front end of the handle, whereby a counterforce can be sensed by a user who displaces the thumb grip backwards when moving the piston backwards for tensioning the thread loop.

6. The device according to claim 5, wherein the thumb grip of the piston has an elongate bracket which is displaceable in an elongate slot along a casing of the handle in its longitudinal direction, whereby the piston is displaceable by means of the thumb grip along the slot backwards towards the spring, furthermore the piston has at its front end said carrier arranged where this carrier consists of the wall which comprises said slot which opens into a hole in the wall of the piston.

7. A method of applying a thread loop around a skin tag by means of the device of claim 1, comprising the steps of:
a thread cartridge is inserted into and connected with its inner end to the carrier at the first end of the piston,
the thread loop at the outer end of the thread cartridge is wrapped around the skin tag,
the piston is displaced backwards from its base position by means of the thumb grip whereby the carrier pulls the thread of the thread loop so that the thread loop is contracted around the skin tag.

8. The method of claim 7, comprising the step of:
the inner end of the thread cartridge comprising a thread is arranged to be detached from the carrier by tilting the handle in relation to the longitudinal direction of the thread cartridge when the thread loop is applied around the skin tag whereby the thread is slackened and the front end of the handle is then removed from the contracted thread loop with the consequence that the inner end of the thread cartridge slides out and detaches from the carrier.

9. The method of claim 8, comprising the steps of:
when loading a thread cartridge in the handle, a knob attached to the inner end of the thread in the thread cartridge is inserted through a hole in a front wall of the piston so that the knob ends up behind the wall,
the thread is lowered into a slot in the wall, whereby the inner end of the thread can be pulled backwards and into the handle when the piston is moved backwards with the thumb grip.

10. The method of claim 9, comprising the steps of:
a cylinder surrounding the thread between the thread loop and the knob is pressed down between wings which form a clamp in the front part of the handle, whereby the cylinder is held in position when a thread cartridge is loaded to the handle,
the rear end of the cylinder is placed in front of a lug arranged next to and behind the clamp, whereby the cylinder surrounding the thread cannot be moved backwards when the carrier of the piston comprising the slot in the wall stretches the thread and thereby automatically contracts the thread loop when the piston is moved backwards by means of the thumb grip,
when the thumb grip is released, the thread is slackened in the thread cartridge, whereby the spring pushes the piston forwards inside the handle, after which the handle is tilted to an angle of 30 to 90 degrees in relation to the longitudinal direction of the thread and removed so that the thread loop slides out of the carrier.
